(19) United States
(12) Patent Application Publication
(10) Pub. No.: US 2016/0324924 A1
(43) Pub. Date: Nov. 10, 2016

(54) COMPOSITIONS AND METHODS FOR TREATMENT OF EXERCISE-INDUCED PULMONARY HEMORRHAGE OR NASOPHARYNGEAL CICATRIX

(71) Applicant: Riddle Institute, Tomball, TX (US)

(72) Inventor: Michael J. Riddle, JR., Houston, TX (US)

(73) Assignee: Riddle Institute, Tomball, TX (US)

(21) Appl. No.: 15/211,893

(22) Filed: Jul. 15, 2016

Related U.S. Application Data
(63) Continuation-in-part of application No. 14/454,304, filed on Aug. 7, 2014.
(60) Provisional application No. 61/863,779, filed on Aug. 8, 2013.

(51) Int. Cl. A61K 38/17; A61K 38/18; A61K 38/30; A61M 15/08; A61K 38/19; A61K 9/00; A61M 11/00; A61K 38/20; A61K 38/57

(52) U.S. Cl. CPC A61K 38/1709; A61K 38/2053; A61K 38/1866; A61K 38/1808; A61K 38/1825; A61K 38/1833; A61K 38/185; A61K 38/30; A61K 38/2073; A61K 38/208; A61K 38/20; A61K 38/1793; A61K 38/177; A61K 38/57; A61K 38/19; A61K 38/195; A61K 9/0078; A61K 9/007; A61M 11/005; A61M 15/08; A61K 35/28

(57) ABSTRACT

Methods, compositions, and medical device systems relating to treating exercise-induced pulmonary hemorrhage (EIPH) and nasopharyngeal cicatrix (NC) in a mammal. For example, one method comprises administering through inhalation a composition comprising a physiologically acceptable carrier and one or more stem cell derived factors, such as stem cell factors secreted by cultured mesenchymal stem cells (MSCs). The mammal may be a horse, dog, camel, or Homo sapiens.

### BACKGROUND OF THE INVENTION

This application is a continuation-in-part of U.S. application Ser. No. 14/454,304, filed Aug. 7, 2014, which claims priority to U.S. Provisional Application No. 61/863,779 filed Aug. 8, 2013.

### FIELD OF THE INVENTION

The present invention relates generally to the field of medical and veterinary treatment of exercise-induced pulmonary hemorrhage (EIPH) or nasopharyngeal cicatrix. More particularly, it concerns the use of compositions comprising one or more stem cell factors in the treatment of EIPH or nasopharyngeal cicatrix.

### DESCRIPTION OF RELATED ART

Exercise-induced pulmonary hemorrhage (EIPH) is a common condition among horses that race at high speeds, such as Thoroughbred and Standardbred race horses. The prevalence of EIPH in race horses is very high. Endoscopic examination of the trachea and bronchi demonstrate a prevalence of 43 to 75% (Pascoe et al., 1981a; Raphel and Soma, 1982; Mason et al., 1983). Evidence of hemosiderophages found in bronchial lavage specimens of race horses suggests that virtually all race horses experience some degree of EIPH.

In addition to horses, a number of other mammals engage in high-intensity physical activity which may lead to EIPH. Examples of such mammals include, but are not limited to, dogs, camels, and human beings.

Regardless of the species of mammal, EIPH causes decreased performance over time because of the pulmonary fibrosis that occurs during the healing process after an episode of EIPH. EIPH is progressive in nature and continues to worsen over time. EIPH is caused by the increased pulmonary capillary pressure during strenuous exercise events combined with the relatively excessive negative pressure generated in the small airways during rapid and deep breathing during strenuous exercise along with inflammation of the airway mucosa caused from the presence of blood. The pressure gradient created during strenuous exercise causes the blood to be pulled across the alveolar-capillary blood-gas barrier membranes into the small airways. When blood enters the small airways, it causes the endothelium of the small airways to become inflamed, which in turn causes the thin tissue membranes that separate the blood from the small airways to become more porous causing more blood to leak into the airways.

The current standard of care for EIPH in race horses is treatment with furosemide (marketed as Lasix by Sanofi S.A., Paris, France). Furosemide lowers the animal's blood pressure, increases the viscosity of its blood, and functions as a diuretic. However, as of 2013, several countries have banned the use of furosemide in the horse racing industry, and it may also be banned in horse racing in the United States in the near future.

Other patents disclose various treatments for EIPH. For example, Blackwater et al. U.S. Pat. Nos. 4,722,334 and 4,955,372, and Russell et al. U.S. Pat. No 6,027,713 are directed to the administration, via inhalation, of a humidified gas stream as a treatment for horses, including a treatment for EIPH.

However, there remains a need for compositions and methods for the treatment of EIPH.

Nasopharyngeal cicatrix (NC) is a condition that has been observed in horses in subtropical regions of the southern United States. The condition first presents as inflammation of the upper respiratory tract of the animal. Over time, the condition may progress to scarring of the upper respiratory tract, which may lead to narrowing of the animal's airways. In extreme cases, the horse may require a tracheostomy in order to breathe.

Risk factors for NC include the animal's age, pasturing of the animal, and summer season. The agent(s) responsible for NC are not yet known.

There exists a need for compositions and methods for the treatment of NC.

### SUMMARY OF THE INVENTION

In one embodiment, the present disclosure provides a method of treating a condition selected from exercise-induced pulmonary hemorrhage (EIPH) or nasopharyngeal cicatrix (NC) in a mammal, comprising: administering through inhalation a composition comprising a physiologically acceptable carrier and one or more stem cell derived factors.

In one embodiment, the present disclosure provides a composition, comprising: a physiologically acceptable carrier, and one or more stem cell derived factors. The amount of one or more stem cell derived factors may be effective to treat exercise-induced pulmonary hemorrhage (EIPH) or nasopharyngeal cicatrix (NC) in a mammal to whom the composition may be administered through inhalation.

In one embodiment, the present disclosure provides a medical device system for administering through inhalation a composition to treat exercise-induced pulmonary hemorrhage (EIPH) or nasopharyngeal cicatrix (NC) in a mammal, comprising: a reservoir configured to store at least one dose of the composition; a nebulizer configured to nebulize said at least one dose of the composition; and a delivery device configured to receive the at least one nebulized dose of the composition from the nebulizer and to deliver the at least one nebulized dose of the composition to at least one of the oral cavity or the nasal cavity of the mammal.

### DESCRIPTION OF ILLUSTRATIVE EMBODIMENTS

The treatment modality described below demonstrates reduced airway inflammation and improved healing of the lung tissue after an episode of EIPH. The reduced inflammation decreases the amount of blood leaked into the airways. The improved healing from the treatment reduces the amount of lung fibrosis caused by the healing process after an episode of EIPH. Both of these mentioned desirable effects may allow racing mammals to remain competitive for longer periods of time because of the prevention of lung damage caused by EIPH.

The treatment modality described below also has been observed to reduce inflammation and cicatrix (scar) formation in the upper respiratory tract of a mammal presenting with NC. The reduced inflammation delays or reduces cicatrix formation. This may bring about reduced morbidity and/or mortality of a mammal, while allowing the mammal to be pastured, especially in summer months in subtropical regions of the southern United States.

In one embodiment, the present disclosure provides a method of treating a condition selected from exercise-induced pulmonary hemorrhage (EIPH) or nasopharyngeal cicatrix (NC) in a mammal, comprising: administering through inhalation a composition comprising a physiologically acceptable carrier and one or more stem cell derived factors.

“Treating” is used herein to refer to one or more of reducing airway inflammation during an episode of EIPH, improving healing of lung tissue after an episode of EIPH, or both. In the context of NC, “treating” is used herein to refer to one or more of reducing airway inflammation of a mammal presenting with NC, reducing scar formation in the respiratory tract of a mammal presenting with NC, or both. In either context, “improving healing” refers to accelerating healing, i.e., taking less time to reach a desired healthy end-state, and/or bringing the lung and/or upper respiratory tract tissue to a healthier final state. (All relative terms herein are in comparison to results seen when no treatment is administered).

By “stem cell derived factor” is meant a compound which is secreted by a mescenchymal stem cell (MSC) when cultured as described below. Though not to be bound by theory, one or more stem cell derived factors may play a role in cell signaling in one or more cell types.

The culturing of MSCs and secretion of one or more stem cell derived factors may be performed as described by Schinkothe et al., Stem Cells and Development 17:199-205 (2008), which is hereby incorporated herein by reference in its entirety. As described by Schinkothe et al., MSCs were obtained from bone marrow aspirates as well as bone marrow from femur and hip head. Before preparing Ficoll-Paque™ PLUS density gradient centrifugation (Amersham Pharmacia Biotech, Uppsala, Sweden), bone marrow was filtered (mesh 70 μm). The first change of medium, consisting of α-minimum essential medium (α-MEM), 20% (vol/vol) fetal calf serum (FCS), 200 μM L-glutamine, 100 U/ml penicillin, and 100 U/ml streptomycin, was prepared 2 days after culturing at 95% humidity and 5% CO2. Cells were used until passage 3. For every passage or experiment, cells were plated at 2000 cells/cm². Medium was changed twice a week.

A cytokine/chemokine array kit (Ray Biotech Inc., Norcross, GA) may be used to detect a panel of 120 secreted cytokines and chemokines. The manufacturer's recommended protocol may be followed. For quantitative analysis of vascular endothelial growth factor (VEGF), interleukin-8 (IL-8), and tumor necrosis factor (TNF), the BD Cytometric Bead Array Flex system (CBA-Flex system) from BD Biosciences (San Diego, Calif.) may be used. The manufacturer's recommended protocol may be followed. For densitometry analysis, gray scale values may be measured using the computer-based system ImageJ Version 1.33 from the National Institutes of Health (NIH). All values may be recalculated as relative intensity in relation to the range between positive and negative control.

MSCs from independent donors were cultured for 3 days. As a control, hematopoietic stem cells (HSCs) as well as the medium used for MSCs in absence of cells were cultured using same conditions. The supernatant of the stem cell culture as well as the medium control were analyzed using the Ray-Biotech cytokine array for analyzing 120 different cytokines and chemokines. By using computer-based densitometry, the relative intensity in relation to the range between positive and negative control was calculated.

By comparing MSC samples with medium controls and also the supernatant of HSCs, a high number of cytokines were secreted specifically by MSCs. For example, TIMP-2, which shows a relative intensity of 1%, was not significantly influenced by HSCs (10%), but it was increased in the supernatant of MSCs up to a mean value of 62%. The factor angiopoetin-2 increased in the supernatant of MSCs up to a mean value of 47%, whereas the relative concentration in HSCs (1%) compared to control (2%) remained unchanged (FIG. 2A). In contrast, other factors were consumed by the cells and therefore decreased in the supernatant.

By comparing all MSC samples with the controls, 44 of the 120 cytokines were secreted in a significant manner based on a significance level of p<0.05. In contrast, 40 of the tested cytokines showed a significant decrease in the supernatant of MSCs. The secreted cytokines can be pooled in different groups. First of all, different factors that are able to prevent the extrinsic apoptosis were secreted by MSCs. Factors such as soluble TRAIL R3, TRAIL R4, or FAS receptor are examples of these. This effect was supported by consuming pro-apoptotic factors like tumor necrosis factor-α (TNF-α or TNF-β). The second group contained factors that are immunosuppressive or factors for which changes result in an immunosuppressive effect. These are factors like interleukin-12 p40 (IL-12 p40), Acrp30, or the soluble IL-2 receptor α. This effect was also supported by decreasing inflammatory factors like IL-1α/β, IL-2, or RANTES. A third group included factors that are able to induce cell proliferation, especially in hematopoietic cells. These are factors like G-CSF, fibroblast growth factor-4 (FGF-4), or FGF-9. Finally, factors that are able to interfere with angiogenesis were secreted by MSCs. These were factors like angiopoietin-2, bFGF, TIMP-1, TIMP-2, or VEGF. In contrast, the HSCs demonstrated only four factors that had shown a significant increase in the cell supernatant. Those significantly increased were IL-16, FGF-7, Dtk, and CTACK.

Factors specifically identified by Schinkothe et al. as having significantly increases concentration within the supernatant (i.e., secreted specifically) by MSCs were adiponectin (Acrp30), Agouti-related peptide (AgRP), angiopoietin-2, basic fibroblast growth factor (bFGF), betacellulin (BTC), epidermal growth factor receptor (EGF-R), first apoptotic signal protein (FAS), fibroblast growth factor (FGF)-4, FGF-9, granulocyte colony stimulating factor (G-CSF), glucocorticoid-induced tumor necrosis factor receptor (GITR), GITR-ligand, chemokine C-X-C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Ra), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1α, MIP-1β, MIP-3β, macrophase stimulating protein (MSP) α, neurotrophin (NT)-4, oncostatin M, osteoprotegerin, phosphatidylinositol-glycan biosynthesis class F (PIGF), sgp130, soluble tumor necrosis factor receptor type 2 (sTNF RII), tissue inhibitor of metalloproteinase (TIMP)-1, TIMP-2, TNF-related apoptosis-inducing ligand (TRAIL) receptor 3 (R3), TRAIL R4, urokinase receptor (uPAR), vascular endothelial growth factor (VEGF), and VEGF-D.

To verify the results of the cytokine array, Schinkothe et al. also performed quantitative analysis of selected factors on an enlarged group of MSC samples. Using the CBA-Flex system, Schinköthe et al. analyzed the concentration of VEGF, IL-8, and TNF in 10 independent MSC samples. After 3 days of cell culture, the MSCs produced a mean value of 5,330 pg/ml of VEGF. The production of IL-8 was a mean value of 345 pg/ml, whereas TNF was not produced by all of the tested samples (FIG. 3).

In summary, data presented by Schinkothe et al. provide an overview about a large range of factors that were secreted by MSCs under cell culture conditions. These data indicate that MSCs demonstrate all previously described functions in cellular interactions without an external stimulus. The cells secreted angiogenic, immunosuppressive, antiapoptotic, and proliferation-stimulating factors.

In one embodiment, the stem cell may be of the same species as the mammal to which the composition may be to be administered. By doing so, the risk of immune responses by the mammal to stem cell derived factors from another species may be reduced.

In one embodiment, the one or more stem cell derived factors selected from the group may consist of adiponectin (Acrp30), Agouti-related peptide (AgRP), angiopoietin-2, basic fibroblast growth factor (bFGF), betacellulin (BTC), epidermal growth factor receptor (EGF-R), first apoptotic signal protein (FAS), fibroblast growth factor (FGF)-4, FGF-9, granulocyte colony stimulating factor (G-CSF), glucocorticoid-induced tumor necrosis factor receptor (GITR), GITR-ligand, chemokine C-X-C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Rα), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1α, MIP-1β, MIP-3β, macrophase stimulating protein (MSP) α, neurotrophin (NT)-4, oncostatin M, osteoprotegerin, phosphatidylinositol-glycan biosynthesis class F (PIGF), sgp130, soluble tumor necrosis factor receptor type 2 (sTNF RII), tissue inhibitor of metalloproteinase (TIMP)-1, TNF-related apoptosis-inducing ligand (TRAIL) receptor 3 (R3), TRAIL R4, urokinase receptor (uPAR), vascular endothelial growth factor (VEGF), and VEGF-D.

In one embodiment, the method may further comprise culturing mesenchymal stem cells (MSCs) in a medium; and separating a first fraction of the medium comprising the one or more stem cell derived factors from a second fraction of the medium comprising the MSCs, prior to administering the composition.

In one embodiment, the method may further comprise combining the one or more stem cell derived factors with the physiologically acceptable carrier in a formulation, prior to administering the composition.

In one embodiment, the composition further comprises one or more stem cells. In such an embodiment, one or more stem cell derived factor(s) of the composition may be secreted by the stem cell(s) before, during, or after administration of the compound.

A quantity of the composition which may contain effective amounts of one or more stem cell derived factors and may be small enough to be delivered in a short time period (e.g. from 1 min to 30 min, such as from 5 min to 15 min), may herein be termed a “dose.”

The physiologically acceptable carrier may be any compound or mixture of compounds that does not react with the one or more stem cell derived factors, that allows inhalation of the composition, and that has no adverse effects on the mammal. Examples of physiologically acceptable carriers include, but are not limited to, water, saline, and buffers with pH and osmolality values that are roughly equivalent to the mammal's serum pH and osmolality, among others.

To facilitate inhalation, it may be desirable to administer the composition in a nebulized or aerosolized form. In one embodiment, the method further comprises nebulizing the composition by at least one of applying at least a partial vacuum to the composition or applying ultrasound to the composition, prior to administering the composition.

As stated above, administration of the composition may be via inhalation. In one embodiment, the composition may be delivered to one or more of the lungs, the bronchi, the trachea, the sinuses, the nasal mucosa, or the oral mucosa of the mammal. As will be apparent to the person of ordinary skill in the art, to deliver the composition to any of those locations, the composition must first be inhaled via at least one of the oral cavity (mouth) or nasal cavity (nose) of the mammal.

Administration may be performed at one or more times before, during, or after the onset of EIPH, or before, during, or after onset of NC. For example, if permissible under the rules of an organization overseeing a competition in which the mammal intends to take part, it may be desirable to administer at least one dose of the composition prior to the mammal's participation in the competition. Such a prior delivery may have a prophylactic benefit.

For another example, it may be desirable to administer one or more doses shortly after intense physical activity by the mammal. Administration in this context may be performed in response to observed symptoms of EIPH or related syndromes (e.g., nosebleed in race horses), but need not be. It may be desirable in this context to administer a first dose, observe one or more symptoms and/or physiological markers (e.g., hemosiderophage level in bronchial lavage samples), and administer a further dose if the observed symptoms and/or physiological markers indicate a lack of efficacy for the first dose.

For yet another example, it may be desirable to administer one or more doses after an acute episode of EIPH has ceased and while the mammal may be healing from that acute episode. By doing so, pulmonary fibrosis that occurs during the healing process may be reduced. This may retard the progress of EIPH over time, thus allowing a mammal engaged in professional physical or athletic performance to extend its career. The precise number of doses administered and the frequency of administration during the healing process may be determined by the person of ordinary skill in the art as a matter of routine experimentation, in light of the present disclosure.

For another example, it may be desirable to administer one or more doses before, during, or after pasturing of the mammal, especially in summer months and/or in subtropical regions, such as subtropical regions of the United States. Administration in this context may be performed in response to observed symptoms of NC (e.g., nasopharyngeal inflammation in pastured horses), but need not be. It may be desirable in this context to administer a first dose, observe one or more symptoms and/or physiological markers, and administer a further dose if the observed symptoms and/or physiological markers indicate a lack of efficacy for the first dose.

Any delivery device configured to allow inhalation of the composition may be used. In one embodiment, administering comprises delivering the composition to the oral cavity of the mammal by an inhaler. “Inhaler” herein refers to a device with a mouthpiece configured for insertion into the mammal's mouth.

In one embodiment, administering comprises delivering the composition to at least one of the oral cavity and the nasal cavity of the mammal by a mask. In one embodiment, wherein the mammal may be a horse, the mask may be a FLEXINEB™ equine nebulizer device (Nortev, Galway, Ireland).

Any mammal which may suffer EIPH or NC may be the subject of the method. In one embodiment, the mammal may be selected from the group consisting of non-human mammals and Homo sapiens. In a further embodiment, wherein the mammal may be a non-human mammal, the non-human mammal may be selected from the group consisting of horses, dogs, and camels. In a particular embodiment, wherein the mammal may suffer and/or be at risk of NC, the mammal may be a horse.

In one embodiment, the present disclosure provides a composition, comprising: a physiologically acceptable carrier, and an amount of each of one or more stem cell derived factors. The amount of one or more stem cell derived factors may be effective to treat EIPH or NC in a mammal to whom the composition may be administered through inhalation.

The physiologically acceptable carrier and the one or more stem cell derived factors may be as described above.

In one embodiment, the present disclosure provides a medical device system for administering through inhalation a composition to treat EIPH or NC in a mammal, comprising: a reservoir configured to store at least one dose of the composition; a nebulizer configured to nebulize said at least one dose of the composition; and a delivery device configured to receive the at least one nebulized dose of the composition from the nebulizer and to deliver the at least one nebulized dose of the composition to at least one of the oral cavity or the nasal cavity of the mammal.

The composition may be as described above.

Any device or component capable of nebulizing the composition may be used. In one embodiment, the nebulizer may be selected from the group consisting of a pump and an ultrasound generator.

The delivery device may be an inhaler or a mask, such as those describe above. In one embodiment, the delivery device may be further configured to conform to at least one of the mouth, the nose, or the snout of the mammal.

As described above, the mammal may be selected from the group consisting of non-human mammals and Homo sapiens. In embodiments wherein the mammal may be a non-human mammal, the non-human mammal may be selected from the group consisting of horses, dogs, and camels.

The medical device system may be of any form, e.g., with separate reservoir, nebulizer, and delivery device, with such devices being modular and interconnectable, and/or with two or more of the reservoir, the nebulizer, and the delivery device contained in a single housing. For example, the FLEXINEB™ device referred to above comprises the reservoir, the nebulizer, and the delivery device contained in a single housing.

### Example 1

### Clinical Veterinary Administration of a Composition to Treat EIPH

A veterinary hospital performed bronchoalveolar lavage on a female horse having undertaken normal exertion. One direct smear, one concentrated smear, and two cytospin preparations made from fluid obtained from a bronchoalveolar lavage were examined. One of the cytospin preparations was stained with Prussian Blue for hemosiderin. The direct and concentrated smears contained only low numbers of cells and the following reflects findings based on the cytospin preparation. The overall cellularity of the sample appeared low with regard to nucleated cells. Low numbers of erythrocytes were found scattered throughout the smears. A few columnar ciliated respiratory cells and a rare goblet cell were found, with a very small amount of mucous in the background. A 300-cell differential of the non-epithelial cell population consisted of approximately 16% neutrophils, 5% small lymphocytes, 1% eosinophils, and 78% macrophages. In addition, a rare well-granulated mast cell was found after the cell differential was performed. The neutrophils appeared mildly to moderately degenerate or poorly preserved, but this may have been a result of the cells being in low-protein fluid. The macrophages were often heavily vacuolated and frequently contained variable amounts of dark brown to blackish-blued globular material. Occasional binucleated macrophages and fewer multinucleated giant cells were observed. Etiologic agents or neoplastic cells were not found in any of the smears. The Prussian Blue stained smear confirmed the presence of hemosiderin in some of the macrophages.

A veterinarian interpreted the results as symptoms of probable mild chronic inflammation with a mild suppurative component; evidence for hemorrhage. While the overall sample cellularity did not appear increased, the cytologic findings suggested a mild chronic inflammatory response with a milder suppurative component. This was based on the findings of occasional binucleated and multinucleated macrophages and the slight elevation in neutrophil proportions, which should be less than 5%. In addition, some of the macrophages contained hemosiderin, which indicated that the patient had experienced hemorrhage. An infectious cause for the neutrophils was not seen, and this milder suppurative component may have been secondary to the hemorrhage.

The horse received a composition in accordance with embodiments herein.

Thereafter, the horse underwent a second bronchoalveolar lavage. Three smears (one pre spun, one post spun and one cytospin preparation) made from the bronchoalveolar wash were examined. The smears were lowly cellular in regards to intact nucleated cells and were minimally hemodiluted in a lightly proteinaceous background with frequently lysed cells and occasionally pink to purple mucinous material. Although only rare cells were present, the majority of intact cells were macrophages with scattered nondegenerate neutrophils and extremely rare lymphocytes. Macrophages were lightly to moderately vacuolated and rarely contained blue pigment (presumed to be hemosiderin). No cytophagia were seen. No infectious agents or neoplastic cells were seen.

A veterinarian concluded that, if the smears were truly representative, then there was no evidence of inflammation present.

In summary, bronchoalveolar lavage before and after treatment indicated that treatment with a composition in accordance with embodiments herein at least partially treated EIPH.

### Example 1

### Clinical Veterinary Administration of a Composition to Treat EIPH

A veterinary hospital performed bronchoalveolar lavage on a female horse having undertaken normal exertion. One direct smear, one concentrated smear, and two cytospin preparations made from fluid obtained from a bronchoalveolar lavage were examined. One of the cytospin preparations was stained with Prussian Blue for hemosiderin. The direct and concentrated smears contained only low numbers of cells and the following reflects findings based on the cytospin preparation. The overall cellularity of the sample appeared low to possibly slightly increased with regard to nucleated cells, and only rare erythrocytes were found. A few columnar ciliated respiratory cells and a rare goblet cell were found, and there was a small amount of mucous in the background. A 300-cell differential of the non-epithelial cell population consisted of approximately 1% neutrophils, 22% small lymphocytes, 3% well-granulated mast cells, and 74% macrophages. The neutrophils appeared mildly degenerate, which was ascribed to the cells having been in low-protein fluid. The macrophages were often highly vacuolated and occasionally contained structures that were consistent for pollen. Occasional binucleated macrophages and rare multinucleated giant cells were observed. Etiologic agents or neoplastic cells were not found in any of the smears. A macrophage stained positive with Prussian Blue.

A veterinarian interpreted the results as symptoms of possible mild chronic inflammation and evidence for very mild hemorrhage. The sample cellularity appeared borderline and may have been very slightly elevated. The predominant cells in this sample were macrophages, and there were low numbers of binucleated and infrequent multinucleated macrophages. This finding suggested that there may be a mild, chronic inflammatory process present. In addition, a macrophage stained lightly positive with Prussian Blue, which indicated that the patient had experienced previous hemorrhage, which was thought to be quite mild.

The horse received a composition in accordance with embodiments herein.

Thereafter, the horse underwent a second bronchoalveolar lavage. Six smears (direct, concentrated, and a cytospin preparation) made from the bronchoalveolar lavage were examined. The smears were lowly cellular in regards to intact nucleated cells and were minimally hemodiluted in a lightly proteinaceous background with frequently lysed cells and occasionally pink to purple mucinous material. Although only rare cells were present, the majority of intact cells were macrophages with scattered nondegenerate neutrophils and extremely rare lymphocytes. Macrophages were lightly to moderately vacuolated and occasionally contained fine to globular blue-green pigment (presumed to be hemosiderin). No cytophagia was seen. No infectious agents or neoplastic cells were seen.

A veterinarian concluded that, if the smears were truly representative, then there was no evidence of inflammation present.

In summary, bronchoalveolar lavage before and after treatment indicated that treatment with a composition in accordance with embodiments herein at least partially treated EIPH.

All of the compositions, methods, and apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions, methods, and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of treating a condition selected from exercise-induced pulmonary hemorrhage (EIPH) or nasopharyngeal cicatrix (NC) in a mammal, comprising:
administering through inhalation a composition comprising a physiologically acceptable carrier and one or more stem cell derived factors selected from the group consisting of adiponectin (Acrp30), Agouti-related peptide (AgRP), angiopoietin-2, basic fibroblast growth factor (bFGF), betacellulin (BTC), epidermal growth factor receptor (EGF-R), first apoptotic signal protein (FAS), fibroblast growth factor (FGF)-4, FGF-9, granulocyte colony stimulating factor (G-CSF), glucocorticoid-induced tumor necrosis factor receptor (GITR), GITR-ligand, chemokine C-X-C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Rα), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1β, MIP-3β, macrophase stimulating protein (MSP) α, neurotrophin (NT)-4, oncostatin M, osteoprotegerin, phosphatidylinositol-glycan biosynthesis class F (PIGF), sgp130, soluble tumor necrosis factor receptor type 2 (sTNF RII), tissue inhibitor of metalloproteinase (TIMP)-1, TIMP-2, TNF-related apoptosis-inducing ligand (TRAIL) receptor 3 (R3), TRAIL R4, urokinase receptor (uPAR), vascular endothelial growth factor (VEGF), and VEGF-D.

2. The method of claim 1, wherein the composition comprises each of the stem cell derived factors from the group.

3. The method of claim 1, further comprising:
culturing mesenchymal stem cells (MSCs) in a medium; and
separating a first fraction of the medium comprising the one or more stem cell derived factors from a second fraction of the medium comprising the MSCs, prior to administering the composition.

4. The method of claim 3, further comprising combining the one or more stem cell derived factors with the physiologically acceptable carrier in a formulation, prior to administering the composition.

5. The method of claim 1, wherein the composition is delivered to one or more of the lungs, the bronchi, the trachea, the sinuses, the nasal mucosa, or the oral mucosa of the mammal.

6. The method of claim 1, wherein the mammal is selected from the group consisting of non-human mammals and Homo sapiens.

7. The method of claim 6, wherein the mammal is a non-human mammal, and the non-human mammal is selected from the group consisting of horses, dogs, and camels.

8. The method of claim 1, wherein administering comprises delivering the composition to the oral cavity of the mammal by an inhaler.

9. The method of claim 1, wherein administering comprises delivering the composition to at least one of the oral cavity and the nasal cavity of the mammal by a mask.

10. The method of claim 1, further comprising nebulizing the composition by at least one of applying at least a partial vacuum to the composition or applying ultrasound to the composition, prior to administering the composition.

11. The method of claim 1, wherein the condition is EIPH.

12. The method of claim 1, wherein the condition is NC.

13. A composition, comprising:
a physiologically acceptable carrier, and one or more stem cell derived factors selected from the group consisting of adiponectin (Acrp30), Agouti-related peptide (AgRP), angiopoietin-2, basic fibroblast growth factor (bFGF), betacellulin (BTC), epidermal growth factor receptor (EGF-R), first apoptotic signal protein (FAS), fibroblast growth factor (FGF)-4, FGF-9, granulocyte colony stimulating factor (G-CSF), glucocorticoid-induced tumor necrosis factor receptor (GITR), GITR-ligand, chemokine C-X-C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Ra), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1α, MIP-1β, MIP-3β, macrophase stimulating protein (MSP) α, neurotrophin (NT)-4, oncostatin M, osteoprotegerin, phosphatidylinositol-glycan biosynthesis class F (PIGF), sgp130, soluble tumor necrosis factor receptor type 2 (sTNF RII), tissue inhibitor of metalloproteinase (TIMP)-1, TIMP-2, TNF-related apoptosis-inducing ligand (TRAIL) receptor 3 (R3), TRAIL R4, urokinase receptor (uPAR), vascular endothelial growth factor (VEGF), and VEGF-D.

14. The composition of claim 13, wherein the composition comprises each of the stem cell derived factors from the group.

15. A medical device system for administering through inhalation a composition to treat exercise-induced pulmonary hemorrhage (EIPH) or nasopharyngeal cicatrix (NC) in a mammal, comprising:
a reservoir configured to store at least one dose of the composition;
a nebulizer configured to nebulize said at least one dose of the composition; and
a delivery device configured to receive the at least one nebulized dose of the composition from the nebulizer and to deliver the at least one nebulized dose of the composition to at least one of the oral cavity or the nasal cavity of the mammal,
wherein the composition comprises a physiologically acceptable carrier, and one or more stem cell derived factors selected from the group consisting of adiponectin (Acrp30), Agouti-related peptide (AgRP), angiopoietin-2, basic fibroblast growth factor (bFGF), betacellulin (BTC), epidermal growth factor receptor (EGF-R), first apoptotic signal protein (FAS), fibroblast growth factor (FGF)-4, FGF-9, granulocyte colony stimulating factor (G-CSF), glucocorticoid-induced tumor necrosis factor receptor (GITR), GITR-ligand, chemokine C-X-C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Ra), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1α, MIP-1β, MIP-3β, macrophase stimulating protein (MSP) a, neurotrophin (NT)-4, oncostatin M, osteoprotegerin, phosphatidylinositol-glycan biosynthesis class F (PIGF), sgp130, soluble tumor necrosis factor receptor type 2 (sTNF RII), tissue inhibitor of metalloproteinase (TIMP)-1, TIMP-2, TNF-related apoptosis-inducing ligand (TRAIL) receptor 3 (R3), TRAIL R4, urokinase receptor (uPAR), vascular endothelial growth factor (VEGF), and VEGF-D.

16. The medical device system of claim 15, wherein the composition comprises each of the stem cell derived factors from the group.

17. The medical device system of claim 15, wherein the nebulizer is selected from the group consisting of a pump and an ultrasound generator.

18. The medical device system of claim 15, wherein the delivery device is further configured to conform to at least one of the mouth, the nose, or the snout of the mammal.

19. The medical device system of claim 15, wherein the mammal is selected from the group consisting of non-human mammals and Homo sapiens.

20. The medical device system of claim 19, wherein the mammal is a non-human mammal, and the non-human mammal is selected from the group consisting of horses, dogs, and camels.

21. The medical device system of claim 15, wherein the reservoir, the nebulizer, and the delivery device are contained in a single housing.
